(19) United States
(12) Patent Application Publication
(10) Pub. No.: US 2007/0009456 A1
(43) Pub. Date: Jan. 11, 2007

(54) Pigmented anhydrous cosmetic compostition

(76) Inventors: Marie-Laure Delacour, Paris (FR); Florence Berry, Villejuif (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/473,198

(22) Filed: Jun. 23, 2006

Related U.S. Application Data
(60) Provisional application No. 60/696,513, filed on Jul. 6, 2005.

(30) Foreign Application Priority Data
Jun. 23, 2005 (FR) 05 51727

(51) Int. Cl. A61K 8/89

(52) U.S. Cl. 424/63; 424/70.12

(57) ABSTRACT

The disclosure relates to an anhydrous cosmetic makeup composition comprising at least one volatile oil, at least one pigment in an amount greater than or equal to 25% by weight of pigments, and at least one oil-thickening agent, the composition having a hardness ranging from 0.04 to 0.5 newtons. The disclosure also relates to a makeup kit comprising two compositions, for example anhydrous compositions, of different color. The disclosure also relates to a method of effacing and/or covering skin color inhomogeneties, such as marks, by applying the anhydrous composition, or of a mixture of two compositions of the makeup kit to the skin.

This application claims benefit of U.S. Provisional Application No. 60/696,513, filed Jul. 6, 2005, the contents of which are incorporated herein by reference. This application also claims benefit of priority under 35 U.S.C. §119 to French Patent Application No. FR 05 51727, filed Jun. 23, 2005, the contents of which are also incorporated herein by reference.

The present disclosure relates to a tinted anhydrous composition for making up the skin of either the human face or body, for example foundations, blushers, eyeshadows and complexion correctors.

Makeup compositions make it possible to cover areas of skin bearing irregularities, for instance marks or depigmented areas.

When the skin bears pigmentary disorders such as marks or hypopigmented or hyperpigmented areas (corresponding, respectively, to lighter or darker areas of the skin), it is necessary for the makeup to have satisfactory covering power in order to efficiently cover all these defects. This covering power should be proportionally greater the more pronounced the defects. In this case, a high content of pigments may be introduced.

However, with a pigment content of more than 20%, the texture of the composition may become pasty and thus difficult to spread. The makeup obtained may not be homogeneous, and areas of different colors can appear on the skin, making the makeup unattractive. Furthermore, the pasty texture is can be uncomfortable for the user during and after the application of the composition to the skin.

Moreover, the high pigment content may accentuate the problems of stability of the composition during its storage over time. Specifically, the pigments can have a tendency to sediment out and a substantial supernatant of oils may appear. The composition then shows phase separation and is no longer homogeneous: it does not allow homogeneous makeup to be obtained.

Thus, there is a need for makeup compositions that can efficiently cover skin color defects (for example marks and hypopigmented or hyperpigmented areas) and that has a texture allowing easy application to the skin.

The inventors have found, surprisingly, that such a composition can be obtained by combining at least one volatile oil, a high content of pigments and at least one oil thickener to adjust the viscosity.

Accordingly, disclosed herein is an anhydrous composition for making up human facial or body skin, allowing very pronounced skin defects to be efficiently and uniformly covered while at the same time maintaining a creamy texture, which is pleasant to use and leading to makeup that shows good staying power (such as fastness of the color and wear-resistance of the covering of the skin defects) and good covering power.

One embodiment of the present disclosure, for example, is an anhydrous cosmetic makeup composition comprising:

i) an oily phase comprising from 10% to 70% by weight, relative to the total weight of the composition, of at least one volatile oil;

ii) a pulverulent phase comprising at least one pigment in an amount of at least 25% by weight, relative to the total weight of the composition;

iii) at least one oil-thickening agent;

the composition having a penetration force ranging from 0.04 to 0.5 newtons.

A second embodiment of the present disclosure is also an anhydrous cosmetic makeup composition comprising:

i) an oily phase comprising from 10% to 70% by weight, relative to the total weight of the composition, of at least one volatile oil;

ii) a pulverulent phase comprising at least one pigment in an amount such that the composition has a covering index of greater than or equal to 20;

iii) at least one oil-thickening agent;

the composition having a penetration force ranging from 0.04 to 0.5 newtons.

Also disclosed herein is a skin makeup kit comprising a first composition and a second composition of a different color.

Another embodiment of the disclosure is also a non-therapeutic skin makeup process comprising the application to the skin of a composition as defined above, or of a mixture of at least two compositions of different colors, such as from the makeup kit as defined above.

Yet, another embodiment of the present disclosure is the use of a composition, or of a mixture of two compositions of different colors, such as from the makeup kit, as defined above, to efface and/or cover skin color inhomogeneities, for instance marks.

Anhydrous Composition

The compositions according to the present disclosure are anhydrous compositions.

The term “anhydrous composition” means a composition comprising less than or equal to 5% by weight of water for exmaple less than or equal to 2% by weight of water, relative to the total weight of the composition. The composition may also be free of water, the water not being added during the preparation of the composition but corresponding to the residual water introduced by the ingredients mixed together.

Penetration Force of the Composition

The compositions according to the present disclosure have a penetration force ranging from 0.04 to 0.5 N, for example ranging from 0.05 to 0.3 N, further for instance ranging from 0.07 to 0.25 N and such as from 0.1 to 0.2 N.

The penetration force (F) is the texture characteristic of the composition in relation with the force required to obtain a given deformation or penetration of the composition. The hardness (d) of the composition is measured in grams with a texturometer. The penetration force is calculated in newtons according to the formula F=d×g (with F in newtons, d in kg and g in m/s²). By taking the acceleration due to gravity g=10 m/s² (rounded up), and by expressing the hardness in grams, the penetration force is determined according to the relationship: F=d/100 (with F in newtons and d in g).

The penetration force of the composition is measured using a TAXT2i texturometer from the company RHEO.

The composition is packaged in a stainless-steel capsule of cylindrical geometry, 60 mm in diameter and 20 mm tall, or in a glass jar of cylindrical geometry 36 mm in diameter and 20 mm tall.

The capsule or the jar is filled to excess with the composition and the surface is levelled off so that it is smooth. The sample is covered with a watchglass to prevent the volatiles from evaporating off.

The penetration force of the packaged composition is measured, after storage for 24 hours, over 10 minutes at 20° C.

The measuring spindle is a Plexiglass cone with an angle of 40°.

The initiation force of the spindle is 0.02 N. The spindle descends towards the packaged composition at a speed of 2 mm/s. The spindle penetrates the composition at a penetration speed of 1 mm/s and to a penetration depth of 5 mm.

Once the spindle has reached the penetration depth, it returns to its initial state at a speed of 2 mm/s.

Three measurements are performed for the same composition at three different places, uniformly distributed and spaced out in the sample. The mean of these three measurements indicates the penetration force of the composition.

Covering Index of the Composition

According to one emodiment of the disclosure, the composition has a covering index of greater than 20, for instance greater than 30, and further for example greater than 100.

The covering index of the composition according to the present disclosure is measured according to the following method:

The composition is spread out to a thickness of 30 μm on Erichsen type 24/5 contrast card comprising a black background and a white background, and the color is measured using a calorimeter, for example of commercial reference CR-300 from the company Minolta.

The color of the composition applied to the contrast card is measured in the following manner:

The values L*_{black}, a*_{black} and b*_{black}, on the one hand, and the values L*_{white}, a*_{white} and b*_{white}, on the other hand, of the composition applied to the contrast card are measured in the CIE calorimetric space using a CR-300 calorimeter.

The difference ΔE between the color of the deposit on the black part of the contrast card and the color of the deposit on the white part of the contrast card is given by:
ΔE=[(a*_{black}−a*_{white})²+(b*_{black}−b*_{white})²+(L*_{black}−L*_{white})²]^{1/2}.

Similar deposits are produced on two other contrast cards and three measurements are taken on each card. The mean of these nine measurements is then calculated.

The covering index is inversely proportional to the variation in color ΔE between the measurements on the black background and on the white background.

The covering index (1/ΔE)×100 of the compositions according to the disclosure is greater than 20, for instance greater than 30, and further for example greater than 100.

The maximum covering index corresponds to a color variation value ΔE=0.

Oily Phase

The anhydrous composition comprises an oily phase comprising at least one volatile oil.

As used herein, the term “oily phase” means all of the oils present in the composition.

In one embodiment, the composition of the present disclosure comprises two volatile oils of different volatility.

As used herein, the term “volatile oil” means any oil that is capable of evaporating on contact with the skin, at room temperature and atmospheric pressure. The volatile oils of the disclosure are volatile cosmetic oils that are liquid at room temperature, with a non-zero vapour pressure at room temperature and atmospheric pressure, ranging for example from 0.13 Pa to 40,000 Pa (0.001 to 300 mmHg), and further for example ranging from 1.3 Pa to 1,300 Pa (0.01 to 10 mmHg).

The at least one volatile oil can be chosen from volatile hydrocarbon-based oils, volatile silicone oils and volatile fluoro oils.

According to one embodiment of the present dislcosure, for example, the volatile oil is chosen from hydrocarbon-based oils.

As used herein, the term “hydrocarbon-based oil” means an oil mainly comprising hydrogen and carbon atoms, and possibly oxygen, nitrogen, sulfur and/or phosphorus atoms.

The volatile hydrocarbon-based oils may be chosen from hydrocarbon-based oils comprising from 8 to 16 carbon atoms, and for example branched C₈-C₁₆ alkanes, for instance C₈-C₁₆ isoalkanes of petroleum origin (also known as isoparaffins), for instance isododecane (also known as 2,2,4,4,6-pentamethylheptane), isohexadecane and, for example, the oils sold under the trade names Isopar® and Permethyl®.

Non-limiting examples of volatile oils that may also be used include volatile silicones, for instance volatile linear or cyclic silicone oils, such as those with a viscosity ≦5 centistokes (5×10⁻⁶ m²/s) and for example comprising from 2 to 10 silicon atoms and from 2 to 7 silicon atoms, these silicones optionally comprising alkyl or alkoxy groups comprising from 1 to 10 carbon atoms. As volatile silicone oils that may be used in the disclosure, non-limiting mention may be made of octamethylcyclotetrasiloxane, decamethylcyclopentasiloxane, dodecamethylcyclohexasiloxane, hepta-methylhexyltrisiloxane, heptamethyloctyltrisiloxane, hexamethyidisiloxane, octamethyltri-siloxane, decamethyltetrasiloxane and dodecamethylpentasiloxane, and mixtures thereof.

The volatile fluoro oil generally does not have a flash point.

Non-limiting examples of volatile fluoro oils that may be mentioned include nonafluoroethoxybutane, nonafluoromethoxybutane, decafluoropentane, tetradecafluoro-hexane and dodecafluoropentane, and mixtures thereof.

The composition comprises at least two volatile hydrocarbon-based oils, such as, for example a mixture of isododecane and of isohexadecane.

The at least one volatile oil may be present in the composition according to the disclosure in an amount ranging from 10% to 70% by weight, such as ranging from 10% to 60% by weight, for example ranging from 10% to 40% by weight, and further for example ranging from 15% to 30% by weight, relative to the total weight of the composition.

The composition according to the disclosure may comprise at least one non-volatile oil.

As used herein, the term “non-volatile oil” means an oil that remains on the skin at room temperature and atmospheric pressure for at least several hours, and for example, has a vapour pressure of less than 0.13 Pa (0.01 mmHg) at room temperature (25° C.).

The optional at least one non-volatile oil may be chosen from hydrocarbon-based oils, for example of animal or plant origin, and silicone oils. As used herein, the term “hydrocarbon-based oil” means an oil mainly comprising hydrogen and carbon atoms and possibly oxygen, nitrogen, sulfur and/or phosphorus atoms.

The non-volatile oils may be chosen from for example hydrocarbon-based oils, which may be fluorinated, and/or non-volatile silicone oils.

Non-limiting examples of non-volatile hydrocarbon-based oils that may mentioned include:
hydrocarbon-based oils of animal origin,
hydrocarbon-based oils of plant origin, such as triglycerides comprising of fatty acid esters of glycerol, the fatty acids of which may have varied chain lengths from C₄ to C₂₄, these chains possibly being linear or branched, and saturated or unsaturated; these oils can be for example heptanoic or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil or musk rose oil; shea butter; or caprylic/capric acid triglycerides, for instance those sold by the company Stéarineries Dubois or those sold under the names Miglyol 810®, 812® and 818® by the company Dynamit Nobel,
synthetic ethers comprising from 10 to 40 carbon atoms,
linear or branched hydrocarbons of mineral or synthetic origin, such as petroleum jelly, polydecenes, hydrogenated polyisobutene such as Parleam®, squalane and liquid paraffins, and mixtures thereof,
synthetic esters, for instance oils of formula R₁COOR₂ wherein R₁ is choosen from linear and branched fatty acid residues comprising from 1 to 40 carbon atoms and R₂ is chosen from a hydrocarbon-based chain, which can be, for example branched, comprising from 1 to 40 carbon atoms, for example where R₁+R₂≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, isodecyl neopentanoate, 2-ethylhexyl palmitate, isostearyl isostearate, 2-hexyldecyl laurate, 2-octyldecyl palmitate, 2-octyldodecyl myristate, or alcohol or polyalcohol heptanoates, octanoates, decanoates or ricinoleates, for instance propylene glycol dioctanoate; hydroxylated esters, for instance isostearyl lactate, diisostearyl malate or 2-octyidodecyl lactate; polyol esters and pentaerythritol esters,
fatty alcohols that are liquid at room temperature with a branched and/or unsaturated carbon-based chain comprising from 12 to 26 carbon atoms, for instance octyldodecanol, isostearyl alcohol, oleyl alcohol, 2-hexyldecanol, 2-butyloctanol or 2-undecylpentadecanol,
higher fatty acids such as oleic acid, linoleic acid or linolenic acid, and mixtures thereof.

The non-volatile silicone oils that may be used in the composition according to the present disclosure may be non-volatile polydimethylsiloxanes (PDMS), polydimethyl-siloxanes comprising alkyl or alkoxy groups, which are pendent and/or at the end of a silicone chain, these groups each comprising from 2 to 24 carbon atoms, phenyl silicones, for instance phenyl trimethicones, phenyl dimethicones, phenyltrimethyl-siloxydiphenylsiloxanes, diphenyl dimethicones and diphenylmethyldiphenyltrisiloxanes, and mixtures thereof.

In one embodiment, the non-volatile oil may be chosen, for example, from C₁₂-C₃₆ esters such as those described above.

The at least one non-volatile oil may be present in the composition according to the disclosure in an amount ranging from 1% to 60% by weight, such as ranging from 3% to 50% by weight, for example ranging from 5% to 35% by weight and further, for example, ranging from 10% to 30% by weight, relative to the total weight of the composition.

The oily phase of the composition according to the disclosure is present in a total amount ranging from 10% to 70% by weight, such as ranging from 10% to 60% by weight, for example ranging from 10% to 40% by weight and further, for example, ranging from 15% to 30% by weight relative to the total weight of the composition.

The compositions according to the present disclousre may also comprise at least one fatty substance other than the oils mentioned above, for instance waxes or pasty fatty substances.

As used herein, the term “waxes” means a fatty substance that is solid at room temperature.

The pasty fatty substances as used herein may be defined by at least one of the following physicochemical properties:
a viscosity ranging from 0.1 to 40 Pa·s (1 to 400 poises), measured at 40° C. with a Contraves TV rotary viscosimeter equipped with a MS-r3 or MS-r4 spindle at a frequency of 60 Hz,
a melting point of 25-70° C., such as 25-55° C.

Among waxes that may be used according to the disclosure, non-limiting mention may be made of:
waxes of animal origin such as beeswax, spermaceti, lanoline wax and lanoline derivatives, plant waxes such as carnauba wax, candelilla wax, ouricury wax, Japan wax, cocoa butter, cork fibre wax or sugar cane wax,
mineral waxes, for example paraffin wax, petroleum jelly wax, lignite wax, microcrystalline waxes or ozokerites,
synthetic waxes, among which are polyethylene waxes, polytetrafluoroethylene waxes and the waxes obtained by Fisher-Tropsch synthesis,
silicon waxes, for example substituted linear polysiloxanes; non-limiting examples that may be mentioned include silicon polyether waxes, alkyl or alkoxy dimethicones comprising from 16 to 45 carbon atoms, and alkyl methicones, for instance the C₃₀-C₄₅ alkyl methicone sold under the trade name “AMS C30” by Dow Corning,
hydrogenated oils that are solid at 25° C., such as hydrogenated castor oil, hydrogenated jojoba oil, hydrogenated palm oil, hydrogenated talo, hydrogenated coconut oil and fatty esters that are solid at 25° C., for instance the C₂₀-C₄₀ alkyl stearate sold under the trade name “Kester Wax K82H” by the company Koster Keunen,
and mixtures thereof.

In one embodiment, for example, polyethylene waxes, microcrystalline waxes, carnauba waxes, hydrogenated jojoba oil, candelilla waxes and beeswaxes, and/or mixtures thereof, may be be used.

The at least one wax, when present, can be present in an amount ranging from 0.05% to 5% by weight, and for example, from 0.1% to 4% by weight, relative to the total weight of the composition.

These fatty substances may be chosen in a varied manner by a person skilled in the art in order to prepare a composition having the desired properties, for example in terms of consistency or texture.

The composition may comprise a fatty phase formed from the oils and fatty substances described above, present in an amount ranging from 10% to 80% by weight, and for instance from 20% to 70% by weight, relative to the total weight of the composition.

Pulverulent Phase

The composition according to the present disclosure comprises a pulverulent phase comprising pigments.

As used herein, the term “pigments” should be understood as meaning mineral or organic particles, which are insoluble in the liquid organic phase, and which are intended to color and/or opacify the composition.

The pigments may be mineral or organic pigments. Pigments that may be used include metal oxides, for instance iron oxides (such as yellow, red, brown and black iron oxides), titanium dioxides, cerium oxide, zirconium oxide and chromium oxide; manganese violet, ultramarine blue, Prussian blue, cobalt blue, ferric blue, bismuth oxychloride, nacre, mica coated with titanium or with bismuth oxychloride, colored nacreous pigments such as titanium mica with iron oxides, titanium mica with, for example, ferric blue or chromium oxide, titanium mica with an organic pigment of the above mentioned type and nacreous pigments based on bismuth oxychloride, and mixtures thereof.

In one embodiment, for example, Iron oxide or titanium oxide pigments may be used.

The pigments may be treated with a hydrophobic agent to make them compatible with the organic phase of the composition. The hydrophobic-treatment agent may be chosen from silicones, for instance methicones, dimethicones or perfluoroalkyl-silanes; fatty acids, for instance stearic acid; metal soaps, for instance aluminium dimyristate, the aluminium salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, and amino acids; N-acylamino acids or salts thereof; lecithin, isopropyl triisostearyl titanate, and mixtures thereof.

The N-acylamino acids may comprise an acyl group comprising from 8 to 22 carbon atoms, for instance a 2-ethylhexanoyl, caproyl, lauroyl, myristoyl, palmitoyl, stearoyl and cocoyl group. The salts of these compounds may be aluminium, magnesium, calcium, zirconium, zinc, sodium and potassium salts. The amino acid may be, for example, lysine, glutamic acid and alanine.

As used herein, the term “alkyl” in the compounds mentioned above means, for example, an alkyl group comprising from 1 to 30 carbon atoms, and such as comprising from 5 to 16 carbon atoms.

Hydrophobic-treated pigments are described for example in European Patent Application No. A-1 086 683.

The at least one pigment is present in the composition according to the present disclosure in an amount greater than or equal to 25% by weight, such as ranging from 25% to 65% by weight, for example ranging from 25% to 60% by weight, further for example ranging from 25% to 55% by weight and such as ranging from 30% to 55% by weight, relative to the total weight of the composition.

Besides pigments, the composition according to the present disclosure may comprise fillers, nacres and liposoluble dyes.

As used herein, the term “fillers” should be understood as meaning colorless or white, mineral or synthetic particles of any form, which are insoluble in the medium of the composition irrespective of the temperature at which the composition is manufactured.

The fillers may be mineral or organic and of any form, platelet-shaped, spherical or oblong, irrespective of the crystallographic form (for example lamellar, cubic, hexagonal, orthorhombic, etc.). Non-limiting mention may be made of talc, mica, silica, kaolin, polyamide (Nylon®) powders, poly-β-alanine powders, polyethylene powders, polymethyl methacrylates, polyurethane powders such as the powder of the copolymer of hexamethylene diisocyanate and of trimethylol hexyl lactone sold under the name Plastic Powder D-400 by the company Toshiki, tetrafluoroethylene polymer (Teflon®) powders, lauroyllysine, starch, boron nitride, polyvinylidene chloride/acrylonitrile copolymers, acrylic acid copolymers, silicon resin powders, for example silsesquioxane powders (silicon resin powders described such as in European Patent No. 293795; for example Tospearls® from Toshiba), elastomeric polyorganosiloxane particles, precipitated calcium carbonate, magnesium carbonate, magnesium hydrogen carbonate, hydroxyapatite, hollow silica microspheres, glass or ceramic microcapsules, and metal soaps derived from organic carboxylic acids comprising from 8 to 22 carbon atoms and for instance from 12 to 18 carbon atoms, for example zinc stearate, magnesium stearate, lithium stearate, zinc laurate or magnesium myristate; barium sulfate, and mixtures thereof.

According to one embodiment of the present disclosure, the compositions comprise at least one filler with a density of less than 0.2 g/cm³.

The density ranges from 0.01 to 0.2 g/cm³ and for example ranges from 0.05 to 0.1 g/cm³.

To obtain this low density, fillers comprising expanded polymers or copolymers based, for example, on acrylonitrile and on an acrylic or styrene monomer and/or on vinylidene chloride may be used.

It is possible to use, for example, a copolymer comprising from 0 to 60% of units derived from vinylidene chloride (such as from 0.1% to 60%), from 20% to 90% of units derived from acrylonitrile and from 0 to 50% of units derived from an acrylic or styrene monomer (such as from 0.1% to 50%), the sum of the percentages (by weight) being equal to 100. The acrylic monomer may be, for example, a methyl or ethyl acrylate or methacrylate. The styrene monomer may be, for, example, α-methylstyrene or styrene.

The fillers with a density of less than 0.2 g/cm³ that may be used in the compositions according to the present disclosure are hollow particles of an expanded copolymer of vinylidene chloride and of acrylonitrile or of vinylidene chloride, of acrylonitrile and of methacrylate.

The particles of the present disclosure may be obtained, for example, according to the processes of European Patent Nos. EP-56 219, EP-348 372, EP-486 080, EP-320 473, EP-112 807 and U.S. Pat. No. 3,615,972.

The inner cavity of the particles in principle comprise a gas that may be air, nitrogen or a hydrocarbon, for instance isobutane or isopentane.

The fillers with a density of less than 0.2 g/cm³ have a granulometry ranging from 1 μm to 80 μm, for example ranging from 10 μm to 50 μm, and further for example from 10 μm to 30 μm.

The fillers with a density of less than 0.2 g/cm³ in the compositions according to the present disclosure may be, for example, expanded terpolymer microspheres of vinylidene chloride, of acrylonitrile and of methacrylate, sold under the brand name Expancel by the company Nobel Casco under the references 551 DE 50 (granulometry of about 40 μm), 551 DE 20 (granulometry of about 30 μm and density of about 65 kg/m³), 551 DE 12 (granulometry of about 12 μm), 551 DE 80 (granulometry of about 80 μm) and 461 DE 50 (granulometry of about 50 μm). Microspheres formed from the same expanded terpolymer with a granulometry of about 18 μm and a density of about 70 kg/m³, referred to hereinbelow as EL 23, or with a granulometry of about 34 μm and a density of about 20 kg/m³, referred to hereinbelow as EL 43, may also be used.

In the compositions of the present disclosure, the at least one filler with a density of less than 0.2 g/cm³, when present, can be present in an amount ranging from 0.05% to 10% by weight, such as from 0.1% to 5% by weight and for example from 0.1% to 3% by weight, relative to the total weight of the composition.

The at least one filler, when present, may be present in each of the compositions according to the disclosure in a total amount ranging from 0.05% to 30% by weight, for example ranging from 0.5% to 15% by weight and further, for example, ranging from 0.5% to 7% by weight relative to the total weight of the composition.

Besides the pigments and fillers, the particulate phase of the disclosure may further comprise at least one nacre and/or dye.

As used herein, the term “nacres” should be understood as meaning iridescent particles, produced for example by certain molluscs in their shell or else synthesized, which are insoluble in the medium of the composition.

The nacres may be chosen from white nacreous pigments such as mica coated with titanium or with bismuth oxychloride, colored nacreous pigments such as titanium mica with iron oxides, titanium mica with, for example, ferric blue or chromium oxide, titanium mica with an organic pigment of the abovementioned type and also nacreous pigments based on bismuth oxychloride.

As used herein, the term “liposoluble dyes” should be understood as meaning generally organic compounds that are soluble in fatty substances such as oils.

The liposoluble dyes can be, for example Sudan red, D&C Red No. 17, D&C Green No. 6, β-carotene, soybean oil, Sudan brown, D&C Yellow No. 11, D&C Violet No. 2, D&C Orange No. 5, quinoline yellow, annatto and bromo acids.

The at least one nacre and/or dye may be present in each of the compositions according to the present disclosure in an amount ranging from 0.05% to 30% by weight, such as ranging from 1% to 20% by weight and for example ranging from 3% to 10% by weight, relative to the total weight of the composition.

According to one embodiment of the present disclosure, the pulverulent phase and the oily phase are present in the composition in a pulverulent phase/oily phase weight ratio ranging from 0.3 to 2, such as from 0.4 to 1.8, for example from 0.5 to 1.5, and further for example from 0.7 to 1.3.

According to yet another embodiment, the oily phase and the pulverulent phase are present in a combined amount of at least 85% by weight, relative to the total weight of the composition.

According to another embodiment, the compositions according to the disclosure are stable for two months at 45° C.

Oil-Thickening Agents

The compositions according to the disclosure comprise at least one oil-thickening agent chosen from polymeric thickeners and mineral thickeners.

The polymeric thickener present in the composition according to the disclosure is an amorphous polymer formed by polymerization of an olefin. The olefin may, for example be an elastomeric ethylenically unsaturated monomer.

Non-limiting examples of olefins that may be mentioned include ethylenic carbide monomers comprising one or two ethylenic unsaturations and comprising from 2 to 5 carbon atoms, such as ethylene, propylene, butadiene or isoprene.

The polymeric thickener is capable of thickening or gelling the organic phase of the composition. As used herein, the term “amorphous polymer” means a polymer that does not have a crystalline form. The polymeric thickener may also be film-forming, i.e. it is capable of forming a film when applied to the skin.

The at least one polymeric thickener may, for example, be chosen from diblock, triblock, multiblock, radial and star copolymers.

Such polymeric thickeners are described in U.S. Patent Application Publication No.2002/005 562 and in U.S. Pat. No. 5,221,534.

In one embodiment of the present disclosure, the at least one polymeric thickener may be an amorphous block copolymer of styrene and of olefin.

The at least one polymeric thickener may be hydrogenated to reduce the residual ethylenic unsaturations after polymerization of the monomers.

The polymeric thickener may be an optionally hydrogenated copolymer comprising styrene blocks and comprising ethylene/C₃-C₄ alkylene blocks.

Among diblock copolymers, which may be, for example, hydrogenated, non-limiting mention may be made of styrene-ethylene/propylene copolymers and styrene-ethylene/butadiene copolymers. Further non-limiting examples of diblock polymers include those sold under the name Kraton® G1701 E by the company Kraton Polymers.

Among triblock copolymers, which may be, for example, hydrogenated, non-limiting mention may be made of styrene-ethylene/propylene-styrene copolymers, styrene-ethylene/butadiene-styrene copolymers, styrene-isoprene-styrene copolymers and styrene-butadiene-styrene copolymers. Triblock polymers are by way of non-limiting example sold under the names Kraton® G1650, Kratono G1652, Kraton@ D1101, Kraton® D1102 and Kraton® D1160 by the company Kraton Polymers.

In one embodiment, a mixture of hydrogenated styrene-butadiene/ethylene-styrene triblock copolymer and of hydrogenated ethylene-propylene-styrene star polymer may also be used, such a mixture, for example, being isododecane. Such mixtures are sold, for example, by the company Penreco under the trade names Versagel® M5960 and Versagel® M5670.

In another embodiment, a diblock copolymer such as those described above, for example a styrene-ethylene/propylene diblock copolymer, may be used as the at least one polymeric thickener.

The at least one polymeric thickener may be present in an amount ranging from 0.1% to 10% by weight, for example ranging from 0.5% to 5% by weight and for example ranging from 1% to 3% by weight, relative to the total weight of the composition.

The composition may also comprise at least one mineral oil-thickening agent such as an organophilic clay or pyrogenic silicas.

Organophilic clays are clays modified with chemical compounds that make the clay able to swell in oily media.

Clays are products that are already well known per se, which are described, for example, in the publication “Minéralogie des argiles [Mineralogy of clays], S. Caillère, S. Hénin, M. Rautureau, 2nd Edition 1982, Masson,” the teaching of which is incorporated herein by way of reference.

Clays are silicates comprising a cation that may be chosen from calcium, magnesium, aluminium, sodium, potassium and lithium cations, and mixtures thereof.

Non-limiting examples of such products that may be mentioned include clays of the smectite family such as montmorillonites, hectorites, bentonites, beidellites and saponites, and also of the vermiculite, stevensite and chlorite families.

These clays may be of natural or synthetic origin. Clays that are cosmetically compatible and acceptable with keratin materials such as the skin may also be used.

The at least one organophilic clay may be chosen from montmorillonite, bentonite, hectorite, attapulgite and sepiolite. In one embodiment of the present disclosure, the at least one clay is a bentonite or a hectorite

These clays may be modified with at least one chemical compound chosen from quaternary amines, tertiary amines, amine acetates, imidazolines, amine soaps, fatty sulfates, alkyl aryl sulfonates and amine oxides.

Non-limiting examples of organophilic clays that may be used include quaternium-18 bentonites such as those sold under the names Bentone 3, Bentone 38 and Bentone 38V by the company Rheox, Tixogel VP by the company United Catalyst, Claytone 34, Claytone 40 and Claytone XL by the company Southern Clay; stearalkonium bentonites such as those sold under the names Bentone 27 by the company Rheox, Tixogel LG by the company United Catalyst and Claytone AF and Claytone APA by the company Southern Clay; quaternium-18/benzalkonium bentonites such as those sold under the names Claytone HT and Claytone PS by the company Southern Clay.

The pyrogenic silicas may be obtained by high-temperature hydrolysis of a volatile silicon compound in an oxhydric flame, producing a finely divided silica. This process makes it possible, for example, to obtain hydrophilic silicas having a large number of silanol groups at their surface. Such hydrophilic silicas are sold, for example, under the names Aerosil 130®, Aerosil 200®, Aerosil 255®, Aerosil 300® and Aerosil 380® by the company Degussa, and Cab-O-Sil HS-5®, Cab-O-Sil EH-5®, Cab-O-Sil LM-130®, Cab-O-Sil MS-55® and Cab-O-Sil M-5® by the company Cabot.

It is possible to chemically modify the surface of the said silica, via a chemical reaction generating a reduction in the number of silanol groups. It is for example possible to substitute silanol groups with hydrophobic groups: a hydrophobic silica is then obtained.

The hydrophobic groups may be chosen from:
trimethylsiloxyl groups, which are obtained for example by treating pyrogenic silica in the presence of hexamethyldisilazane. Silicas thus treated are known as “silica silylate” according to the CTFA (6th Edition, 1995). They are sold, for example, under the references Aerosil R812® by the company Degussa and Cab-O-Sil TS-530® by the company Cabot;
dimethylsilyloxyl or polydimethylsiloxane groups, which are obtained for example by treating pyrogenic silica in the presence of polydimethylsiloxane or dimethyidichlorosilane. Silicas thus treated are known as “silica dimethyl silylate” according to the CTFA (6th Edition, 1995). They are sold, for example, under the references Aerosil R972® and Aerosil R974® by the company Degussa and Cab-O-Sil TS-610® and Cab-O-Sil TS-720® by the company Cabot.

The pyrogenic silica may have a particle size that may be nanometric to micrometric, for example ranging from about 5 to 200 nm.

The at least one mineral oil-thickening agent may be present in the composition in an amount ranging from 0.5% to 7% by weight, such as in an amount ranging from 1% to 5% by weight, and for example ranging from 1% to 3% by weight, relative to the total weight of the composition.

The at least one oil-thickening agent may be present in the composition in a total amount ranging from 0.1% to 10% by weight, such as ranging from 1% to 7% by weight and for instance ranging from 2% to 6% by weight, relative to the total weight of the composition.

The composition according to the disclosure may comprise at least one other common cosmetic ingredient, which may be chosen, for example, from antioxidants, fragrances, preserving agents, neutralizers, surfactants, sunscreens, vitamins, moisturizers, self-tanning compounds, anti-wrinkle active agents, emollients, hydrophilic or lipophilic active agents, free-radical scavengers, sequestrants and film-forming agents.

The composition described above has a texture that allows two compositions in accordance with the disclosure, and of different color, to be mixed together easily. Thus, it is possible to produce a makeup kit comprising at least one composition, and in one embodiment, two compositions, according to the disclosure, allowing the user to prepare novel shades obtained according to varied weight mixtures using each composition of the kit.

Another embodiment of the present disclosure is also a skin makeup kit comprising:

i) a first composition comprising at least one pigment and at least one volatile oil, the first composition having a hue angle h ranging from 40° to 72°;

ii) a second composition of different color from the first, the second composition comprising at least one pigment and at least one volatile oil, the second composition having a hue angle h ranging from 40° to 72°;

wherein the first and second compositions have a difference in hue angle h ranging from 4° to 40°.

According to another embodiment of the present disclosure, the first composition of the kit has a hue angle value h ranging from 40° to 60°, for example ranging from 42° to 56° and further for example ranging from 42° to 52°; and the second composition of the kit has a hue angle value h ranging from 54° to 72°, for example from 56° to 68° and further for example ranging from 58° to 68°.

According to yet another embodiment, the first and second compositions have a difference in hue angle h for example ranging from 10° to 30°, and further for example from 15° to 25°.

Also disclosed herein is a skin makeup kit comprising:

i) a first composition comprising at least one pigment and at least one volatile oil, the first composition having a lightness L* ranging from 30 to 80;

ii) a second composition of different color from the first, the second composition comprising at least one pigment and at least one volatile oil, the second composition having a lightness L* ranging from 30 to 80;

wherein the first and second compositions having a difference in lightness ΔL* ranging from 2 to 50.

According to another embodiment, the first composition of the kit according to the to the disclosure has a lightness value L* ranging from 30 to 60, for example ranging from 35 to 60, and further for example ranging from 40 to 60; and the second composition of the kit according to the disclosure has a lightness value L* ranging from 60 to 80, for instance ranging from 60 to 76, and further for example ranging from 60 to 70.

According to yet another embodiment of the disclosure, the first and second compositions have a difference in lightness ΔL* , for example, ranging from 2 to 40, and further for example from 20 to 30.

Also disclosed herein is a skin makeup kit comprising:

i) a first composition comprising at least one pigment and at least one volatile oil, the first composition having a saturation C* ranging from 10to 40;

ii) a second composition of different color from the first, the second composition comprising at least one pigment and at least one volatile oil, the second composition having a saturation C* ranging from 10 to 40;

wherein the first and second compositions have a difference in saturation ΔC* ranging from 0.1 to 30.

According to one embodiment of the present disclosure, the first composition of the kit has a saturation value C* ranging from 10 to 25, such as ranging from 15 to 25, and for example ranging from 20 to 25; and the second composition of the kit has a saturation value C* ranging from 25 to 40, such as ranging from 25 to 35, and for example ranging from 25 to 31.

According to another embodiment of the present disclosure, the first and second compositions have a difference in saturation ΔC*, for example, ranging from 5 to 25, and further for example from 5 to 20.

The compositions corresponding to the colorimetric parameters defined above can be, for example, obtained by mixing pigments, and optionally fillers, nacre, organic dyes and other ingredients.

The hue angle h, lightness L* and saturation C* colorimetric parameters are measured according to the method described below.

The difference in hue angle Δh corresponds to the absolute value of the difference between the hue angle h of the first composition of the kit and that of the second composition.

Similarly, the differences in lightness ΔL* and in saturation ΔC* correspond, respectively, to the difference (as an absolute value) in lightness L* and in saturation C* between the first composition and the second composition of the kit.

According to one embodiment of the present disclosure, the makeup kit defined above is such that at least one from among the first and second compositions is an anhydrous composition as defined in the present disclosure.

According to another embodiment of the present disclosure, the makeup kit is such that both the first and second compositions are in accordance with the anhydrous compositions described in the present patent application.

According to yet another embodiment of the present disclosure, the compositions are presented in the form of a kit comprising two compartments wherein the two compositions are separately packaged.

According to still another embodiment, the two compartments may be delimited by separate containers, for example in the form of tubes, packaged, for instance, in the same packaging such as a case or a blister pack.

The compartments may be coupled to each other via their respective lids, or may be formed from the same component. According to this embodiment, the tubes are aligned along the same axis and are arranged so as to have their respective apertures facing each other. The compositions of the kit according to thedisclosure may also be packaged in two pump-dispenser bottles that are actuated together or separately to allow better metering of the two compositions, or alternatively in the form of two heating bags with a foam or fine brush tip, to facilitate the mixing of the two shades.

In one embodiment, for example, tubes that combine ease of metering and reduced cost are chosen.

Other packaging forms may be used to couple the compartments together, such as, for example, moulding within the same component, or by any other system of fastening, such as by click-fastening or bonding.

Method for Measuring the Colorimetric Parameters of a Composition in the Bulk

The equipment used is as follows:
Galvanized steel crucibles (30-40 mm×20-30 mm×2 mm)
Glass slides (75×25 mm)
Minolta CM 3610d spectrophotometer
Quality control software
   Preparation of the Sample

The crucible is filled with bulk until the composition overflows slightly. A glass slide is placed over the filled crucible, while checking that no air bubbles have formed. It is necessary for the appearance of the product under the slide to be perfectly smooth and homogeneous for the measurement.

Measurement of the Sample

To measure the colorimetric parameters (L*, C* and h) of the prepared sample of composition, a measurement of the color in the bulk product is performed using a Minolta® CM 3610d spectrocolorimeter with a mean aperture of 12 mm, an angle of 8°, and with specular reflection mode included.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples are intended to illustrate the present disclosure in a non-limiting manner.

### EXAMPLE 1

### Anhydrous Foundation

An anhydrous foundation having the composition below was prepared:
Isohexadecane 4.2 g
Cyclopentasiloxane 15.0 g
Isododecane 5.0 g
Isononyl isononanoate 5.4 g
Iron oxides (yellow, black and brown) 9.1 g
Titanium oxides 35.9 g
Hectorite modified with distearyl dimethyl ammonium chloride at 10% in petroleum distillate, sold under the name Bentone Gel SS 71 V by the company Elementis 15 g
Styrene/ethylene-propylene diblock copolymer sold under the name Kraton G1701E by the company Kraton Polymers 2.1 g
Sorbitan stearate 1.3 g
Kaolin 5.0 g
Crosslinked polydimethylsiloxane sold under the name DC 9506 by the company Dow Corning 0.5 g
Vinylidene chloride/acrylonitrile/PMMA microspheres expanded with isobutane, sold under the name Expancel 551 DE 20 D60 by the company Expancel 1.5 g

The weight ratio between the pulverulent phase and the oily phase was 1.22.

The oils, the Kraton G1701E and the sorbitan stearate were mixed together. The Bentone Gel SS 71 V was dispersed in the gel obtained above. The pulverulent materials: first the dyestuffs (pigments, dyes and nacres) and then the fillers, were then dispersed in this mixture.

The foundation obtained had a soft texture (penetration force F=0.25 N±0.05 N), was soft and applied easily to the skin with good sliding power.

This foundation had a coverage index of 200 measured according to the protocol described previously.

The makeup obtained was homogeneous and comfortable, and left on the skin a uniform deposit that gave very good covering of color imperfections. Moreover, the makeup showed good color fastness and wear-resistance in the covering of the skin defects in the course of the day.

### EXAMPLE 2

### Anhydrous Foundation

A foundation having the composition below was prepared:
Isohexadecane 15 g
Isododecane 12 g
Isononyl isononanoate 16 g
Iron oxides (yellow, black and brown) 5 g
Titanium oxides 20 g
Hectorite modified with distearyl dimethyl ammonium chloride at 10% in petroleum distillate, sold under the name Bentone Gel SS 71 V by the company Elementis 20 g
Styrene/ethylene-propylene diblock copolymer sold under the name Kraton G1701 E by the company Kraton Polymers 2.5 g
Sorbitan stearate 1.5 g
Nylon powder (orgasol 2002 EXD NAT COS 204 from the company Arkema) 4 g
Talc 3.5 g
Vinylidene chloride/acrylonitrile/PMMA microspheres expanded with isobutane, sold under the name Expancel 551 DE 20 D60 by the company Expancel 0.5 g

The weight ratio between the pulverulent phase and the oily phase was 0.55.

The mode of preparation was the same as that of Example 1.

The foundation obtained had a soft texture (the penetration force F was ranging from 0.04 to 0.5 N) and spread well.

The makeup obtained was homogeneous, covered skin color imperfections, and was comfortable for the user.

### EXAMPLE 3

### Anhydrous Foundation

An anhydrous foundation having the composition below was prepared:
Isohexadecane 15.2 g
Isododecane 11.5 g
Isononyl isononanoate 6.4 g
Iron oxides (yellow, black and brown) 10.71 g
Titanium oxides 34.29 g
Hectorite modified with distearyl dimethyl ammonium chloride at 10% in petroleum distillate, sold under the name Bentone Gel SS 71 V by the company Elementis 15 g
Styrene/ethylene-propylene diblock copolymer sold under the name Kraton G1701E by the company Kraton Polymers 2.1 g
Sorbitan stearate 1.3 g
Nylon powder 2 g
Vinylidene chloride/acrylonitrile/PMMA microspheres expanded with isobutane, sold under the name Expancel 551 DE 20 D60 by the company Expancel 1.5 g

The weight ratio between the pulverulent phase and the oily phase was 1.05.

The mode of preparation was the same as that of Example 1.

The foundation obtained had a soft texture (penetration force F=0.15 N), was gentle and applied easily to the skin with good sliding power.

This foundation had a coverage index of 200 measured according to the protocol described previously.

The makeup obtained was homogeneous and comfortable, and left a uniform deposit on the skin that gave very good covering of color and relief imperfections. Moreover, the makeup showed good colorfastness and wear-resistance in the covering of skin defects in the course of the day.

### EXAMPLE 4

### Anhydrous Foundation

A foundation having the composition below was prepared:
Isohexadecane 15 g
Isododecane 12 g
Isononyl isononanoate 17.5 g
Iron oxides (yellow, black and brown) 25 g
Titanium oxides 5 g
Hectorite modified with distearyl dimethyl ammonium chloride at 10% in petroleum distillate, sold under the name Bentone Gel SS 71 V by the company Elementis 18 g
Styrene/ethylene-propylene diblock copolymer sold under the name Kraton G1701 E by the company Kraton Polymers 2.5 g
Sorbitan stearate 1.5 g
Nylon powder 2g
Pyrogenic silica (Aerosil 200 from the company Degussa-Hüls) 0.5 g
Vinylidene chloride/acrylonitrile/PMMA microspheres expanded with isobutane, sold under the name Expancel 551 DE 20 D60 by the company Expancel 1 g

The weight ratio between the pulverulent phase and the oily phase was 0.56.

The mode of preparationwas the same as that of Example 1.

The foundation obtained had a soft texture (the penetration force F ranges from 0.04 to 0.5 N) and spread well.

The makeup obtained was homogeneous, covered skin color imperfections, and was comfortable for the user.

### EXAMPLE 5

### Anhydrous Foundation

A foundation having the composition below was prepared:
Isohexadecane 8.2 g
Isododecane 10 g
Isononyl isononanoate 8.4 g
Iron oxides (yellow, black and brown) 15g
Titanium oxides 40 g
Hectorite modified with distearyl dimethyl ammonium chloride at 10% in petroleum distillate, sold under the name Bentone Gel SS 71 V by the company Elementis 13 g
Styrene/ethylene-propylene diblock copolymer sold under the 2.1 g name Kraton G1701 E by the company Kraton Polymers
Sorbitan stearate 1.3 g
PMMA 2 g

The weight ratio between the pulverulent phase and the oily phase was 1.5.

The mode of preparation was the same as that of Example 1.

The foundation obtained had a soft texture (the penetration force F ranged from 0.04 to 0.5 N) and spread well.

The makeup obtained was homogeneous, covered skin color imperfections, and was comfortable for the user.

### EXAMPLE 6

### Anhydrous Foundation

A foundation having the composition below was prepared:
Isohexadecane 8.2 g
Isododecane 10 g
Isononyl isononanoate 8.4 g
Cyclopentasiloxane 8.2 g
Iron oxides (yellow, black and brown) 17 g
Titanium oxides 18 g
Hectorite modified with distearyl dimethyl ammonium chloride at 10% in petroleum distillate, sold under the name Bentone Gel SS 71 V by the company Elementis 18 g
Styrene/ethylene-propylene diblock copolymer sold under the name Kraton G1701 E by the company Kraton Polymers 2.5 g
Sorbitan stearate 1.5 g
PMMA 2g
Talc 3g
Nylon 3 g
Vinylidene chloride/acrylonitrile/PMMA microspheres expanded with isobutane, sold under the name Expancel 551 DE 20 D60 by the company Expancel 0.2 g

The weight ratio between the pulverulent phase and the oily phase was 0.86.

The mode of preparation was the same as that of Example 1.

The foundation obtained had a soft texture (the penetration force F ranged from 0.04 to 0.5 N) and spread well.

The makeup obtained was homogeneous, covered skin color imperfections, and was comfortable for the user.

### EXAMPLE 7

### Anhydrous Foundation

A foundation having the composition below was prepared:
Isohexadecane 8.5 g
Isododecane 10 g
Isononyl isononanoate 11 g
Iron oxides (yellow, black and brown) 15 g
Titanium oxides 25 g
Hectorite modified with distearyl dimethyl ammonium chloride at 10% in petroleum distillate, sold under the name Bentone Gel SS 71 V by the company Elementis 18 g
Styrene/ethylene-propylene diblock copolymer sold under the name Kraton G1701 E by the company Kraton Polymers 2.5 g
Sorbitan stearate 1.5 g
PMMA 2g
Talc 3g
Nylon 3g
Vinylidene chloride/acrylonitrile/PMMA microspheres expanded with isobutane, sold under the name Expancel 551 DE 20 D60 by the company Expancel 0.2 g
Silica 0.3 g

The weight ratio between the pulverulent phase and the oily phase was 1.07.

The mode of preparation was the same as that of Example 1.

The foundation obtained had a soft texture (the penetration force F ranged from 0.04 to 0.5 N) and spread well.

The makeup obtained was homogeneous, covered skin color imperfections, and was comfortable for the user.

### EXAMPLE 8

### Anhydrous Foundation

A foundation having the composition below was prepared:
Isohexadecane 11.5 g
Isododecane 12 g
Isononyl isononanoate 16 g
Iron oxides (yellow, black and brown) 10 g
Titanium oxides 25 g
Hectorite modified with distearyl dimethyl ammonium chloride at 10% in petroleum distillate, sold under the name Bentone Gel SS 71 V by the company Elementis 18 g
Styrene/ethylene-propylene diblock copolymer sold under the name Kraton G1701 E by the company Kraton Polymers 2.5 g
Sorbitan stearate 1.5 g
Nylon 2 g
Vinylidene chloride/acrylonitrile/PMMA microspheres expanded with isobutane, sold under the name Expancel 551 DE 20 D60 by the company Expancel 1.5 g

The weight ratio between the pulverulent phase and the oily phase was 0.72.

The mode of preparation was the same as that of Example 1.

The foundation obtained had a soft texture (the penetration force F ranged from 0.04 to 0.5 N) and spread well.

The makeup obtained was homogeneous, covered skin color imperfections, and was comfortable for the user.

### EXAMPLES 9, 10, 11, 12 AND 13

Using the composition of Example 3, by modifying the proportion of titanium dioxide and of the yellow, black and brown iron oxides, 5 anhydrous compositions having the pigmentary compositions below were prepared and the hue angle h, lightness L* and saturation C* values thereof were measured:

| | | | | Example | Example |
|---|---|---|---|---|---|
| Mass % | Example 9 | Example | Example | 12 | 13 |
| in the | Mid- | 10 | 11 | Pale | Dark |
| formulation | Yellow | Mid-Pink | Dark Red | Yellow | Yellow |
| Titanium | 34.29 | 37.71 | 0 | 38.52 | 0 |
| dioxide | | | | | |
| Black iron | 0.36 | 0.4 | 5.18 | 0.38 | 0.96 |
| oxide | | | | | |
| Yellow iron | 9 | 3.71 | 10.85 | 5.07 | 41.28 |
| oxide | | | | | |
| Brown iron | 1.35 | 3.18 | 28.97 | 1.03 | 2.76 |
| oxide | | | | | |
| L*, lightness | 64.96 | 62.69 | 40.43 | 67.92 | 51.98 |
| C*, | 23.25 | 19.75 | 21.83 | 19.21 | 30.9 |
| saturation | | | | | |
| h, hue angle | 65.09° | 47° | 42.14° | 65.4° | 65.97° |

### EXAMPLE 14

### Makeup Kits

A makeup kit comprising the composition of Example 9 (first composition) and the composition of Example 10 (second composition) was prepared.

Each composition was packaged separately in a tube.

The two compositions were mixed together in a 50/50 weight ratio, thus leading to a composition C whose colorimetric characteristics were as follows: L*=64.02, C*=23.40 and h=60.26°.

### EXAMPLE 15

### Eyeshadow

An eyeshadow having the composition below was prepared:
Isohexadecane 15.2g
Isododecane 11.5 g
Isononyl isononanoate 6.4 g
Iron oxides 10 g
Titanium oxides 5 g
Lakes 5 g
Nacres (mica/titanium or mica/titanium/iron oxides) 25 g
Hectorite modified with distearyl dimethyl ammonium chloride at 10% in petroleum distillate, sold under the name Bentone Gel SS 71 V by the company Elementis 15 g
Styrene/ethylene-propylene diblock copolymer sold under the name Kraton G1701 E by the company Kraton Polymers 2.1 g
Sorbitan stearate 1.3 g
Nylon powder 2g
Talc 1.5 g

The weight ratio between the pulverulent phase and the oily phase was 1.05.

The method for preparing this eyeshadow was the same as that for the foundation of Example 1.

The eyeshadow obtained had a soft texture and spread well.

The makeup obtained was homogeneous, had a very strong color that lasted throughout the day, and was comfortable for the user.

### EXAMPLE 16

### Blusher

A blusher having the composition below was prepared:
Isohexadecane 15 g
Isododecane 12 g
Isononyl isononanoate 16 g
Iron oxides 5 g
Titanium oxides 5 g
Lakes 5g
Nacres (mica/titanium) 10 g
Hectorite modified with distearyl dimethyl ammonium chloride at 10% in petroleum distillate, sold under the name Bentone Gel SS 71 V by the company Elementis 20 g
Styrene/ethylene-propylene diblock copolymer sold under the name Kraton G1701 E by the company Kraton Polymers 2.5 g
Sorbitan stearate 1.5 g
Nylon powder 4 g
Talc 3.5 g
Vinylidene chloride/acrylonitrile/PMMA microspheres expanded with isobutane, sold under the name Expancel 551 DE 20 D60 by the company Expancel 0.5 g

The weight ratio between the pulverulent phase and the oily phase was 0.55.

The method for preparing this blusher was the same as that for the foundation of Example 1.

The blusher obtained had a soft texture and spread well.

The makeup obtained was homogeneous, had a strong color that lasted throughout the day, and was comfortable for the user.

What is claimed is:

1. An anhydrous cosmetic makeup composition comprising:
i) an oily phase comprising from 10% to 70% by weight, relative to the total weight of the composition, of at least one volatile oil;
ii) a pulverulent phase comprising at least one pigment in an amount greater than or equal to 25% by weight, relative to the total weight of the composition; and
iii) at least one oil-thickening agent;
wherein the composition has a hardness ranging from 0.04 to 0.5 newtons.

2. A cosmetic composition according to claim 1 wherein the hardness of the composition ranges from 0.1 N to 0.2 N.

3. A cosmetic composition according to claim 1 further comprising at least two volatile oils of different volatility.

4. A cosmetic composition according to claim 1 wherein the at least one volatile oil is chosen from volatile silicone oils and volatile hydrocarbon-based oils.

5. A cosmetic composition according to claim 1 wherein the at least one volatile oil is chosen from volatile hydrocarbon-based oils comprising from 8 to 16 carbon atoms; linear or cyclic silicone oils comprising from 2 to 7 silicon atoms, the silicones optionally comprising alkyl or alkoxy groups comprising from 1 to 10 carbon atoms; and volatile fluoro oils.

6. A cosmetic composition according to claim 5 wherein the at least one volatile oil is chosen from isododecane, isodecane, isohexadecane, cyclopentasiloxane, octamethylcyclotetrasiloxane, decamethylcyclopentasiloxane, dodecamethylcyclo-hexasiloxane, heptamethylhexyltrisiloxane, heptamethyloctyltrisiloxane, hexamethyidisiloxane, octamethyltrisiloxane, decamethyltetrasiloxane and dodecamethylpentasiloxane.

7. A cosmetic composition according to claim 1 comprising at least one volatile silicone oil and at least one volatile hydrocarbon-based oil.

8. A cosmetic composition according to claim 7 comprising isohexadecane and isododecane.

9. A cosmetic composition according to claim 1 wherein the at least one volatile oil is present in an amount ranging from 10% to 60% by weight, relative to the total weight of the composition.

10. A cosmetic composition according to claim 9 wherein the at least one volatile oil is present in an amount ranging from 15% to 30% by weight, relative to the total weight of the composition.

11. A cosmetic composition according to claim 1 wherein the oily phase is present in a total amount ranging from 10% to 70% by weight, relative to the total weight of the composition.

12. A cosmetic composition according to claim 11 wherein the oily phase is present in a total amount ranging from 15% to 30% by weight, relative to the total weight of the composition.

13. A cosmetic composition according to claim 1 wherein the at least one pigment is chosen from mineral pigments.

14. A cosmetic composition according to claim 13 wherein the mineral pigments are chosen from iron oxide and titanium dioxide pigments.

15. A cosmetic composition according to claim 1 wherein the at least one pigment is present in an amount ranging from 25% to 65% by weight, relative to the total weight of the composition.

16. A cosmetic composition according to claim 15 wherein the at least one pigment is present in an amount ranging from 30% to 55% by weight, relative to the total weight of the composition.

17. A cosmetic composition according to claim 1 comprising at least one filler.

18. A cosmetic composition according to claim 17 wherein the at least one filler is chosen from talc, mica, silica, kaolin, polyamide powders, poly-β-alanine powders, polyethylene powders, polymethyl methacrylates, polyurethane powders, tetrafluoroethylene polymer powders, lauroyl lysine, starch, boron nitride, polyvinylidene chloride/acrylonitrile copolymer powders, acrylic acid copolymer powders, silicone resin powders, elastomeric polyorganosiloxane particles, precipitated calcium carbonate, magnesium carbonate, magnesium hydrogen carbonate, hydroxyapatite, hollow silica microspheres, glass and/or ceramic microcapsules, metal soaps derived from organic carboxylic acids comprising from 8 to 22 carbon atoms, and barium sulfate.

19. A cosmetic composition according to claim 1 further comprising at least one filler with a density of less than 0.2 g/cm³.

20. A cosmetic composition according to claim 19 wherein the at least one filler with a density of less than 0.2 g/cm³ is chosen from hollow polyvinylidene chloride/acrylonitrile microspheres and hollow acrylic acid copolymer microspheres.

21. A cosmetic composition according to claim 17 wherein the at least one filler is present in a total amount ranging from 0.05% to 30% by weight, relative to the total weight of the composition.

22. A cosmetic composition according to claim 21 wherein the at least one filler is present in a total amount ranging from 0.5% to 7% by weight, relative to the total weight of the composition.

23. A cosmetic composition according to claim 1 wherein the weight ratio between the pulverulent phase and the oily phase ranges from 0.3 to 2, the oily phase and the pulverulent phase being present in a combined amount greater than or equal to 85% by weight, relative to the total weight of the composition.

24. A cosmetic composition according to claim 23.wherein the weight ratio between the pulverulent phase and the oily phase ranges from 0.7 to 1.3.

25. A cosmetic composition according to claim 1 wherein the oily phase and the pulverulent phase are present in a combined amount of at least 90% by weight, relative to the total weight of the composition.

26. A cosmetic composition according to claim 1 wherein the at least one oil-thickening agent is chosen from polymeric thickeners and mineral thickeners.

27. A cosmetic composition according to claim 26 wherein the at least one polymeric thickener is formed by polymerization of ethylenic carbide monomers comprising at least one ethylenic unsaturation and from 2 to 5 carbon atoms.

28. A cosmetic composition according to claim 26 wherein the at least one polymeric thickener is formed by polymerization of an olefin chosen from ethylene, propylene, butadiene and isoprene.

29. A cosmetic composition according to claim 26 wherein the at least one polymeric thickener is an optionally hydrogenated copolymer comprising styrene blocks and ethylene/C₃-C₄ alkylene blocks.

30. A cosmetic composition according to claim 26 wherein the at least one polymeric thickener is a mixture of hydrogenated styrene-butylene/ethylene-styrene triblock copolymer and of hydrogenated styrene-propylene-styrene star polymer.

31. A cosmetic composition according to claim 26 wherein the at least one polymeric thickener is chosen from styrene-ethylene/propylene or styrene-ethylene/butadiene diblock copolymers, and styrene-ethylene/butadiene-styrene, styrene-isoprene-styrene and styrene-butadiene-styrene triblock copolymers.

32. A cosmetic composition according to claim 26 wherein the at least one polymeric thickener is present in the composition in an amount ranging from 0.1% to 10% by weight, relative to the total weight of the composition.

33. A cosmetic composition according to claim 32 wherein the at least one polymeric thickener is present in the composition in an amount ranging from 1% to 3% by weight, relative to the total weight of the composition.

34. A cosmetic composition according to claim 26 wherein the at least one mineral oil-thickening agent is chosen from organophilic clays and pyrogenic silicas.

35. A cosmetic composition according to claim 26 wherein the at least one mineral oil-thickening agent is present in an amount ranging from 0.5% to 7% by weight, relative to the total weight of the composition.

36. A cosmetic composition according to claim 26 wherein the at least one mineral oil-thickening agent is present in an amount ranging from 1% to 3% by weight, relative to the total weight of the composition.

37. A cosmetic composition according to claim 1 wherein the at least one oil-thickening agent is present in the composition in a total amount ranging from 0.1% to 10% by weight, relative to the total weight of the composition.

38. A cosmetic composition according to claim 37 wherein the at least one oil-thickening agent is present in the composition in a total amount ranging from 2% to 6% by weight, relative to the total weight of the composition.

39. A cosmetic composition according to claim 1 further comprising at least one cosmetic ingredient chosen from antioxidants, fragrances, preserving agents, neutralizers, surfactants, sunscreens, vitamins, moisturizers, self-tanning compounds, anti-wrinkle active agents, emollients, hydrophilic or lipophilic active agents, free-radical scavengers, sequestrants and film-forming agents.

40. An anhydrous cosmetic makeup composition comprising:
i) an oily phase comprising from 10% to 70% by weight, relative to the total weight of the composition, of at least one volatile oil;
ii) a pulverulent phase comprising at least one pigment in an amount such that the composition has a covering index of greater than or equal to 20; and
iii) at least one oil-thickening agent;
wherein the composition has a hardness ranging from 0.04 to 0.5 newtons.

41. A cosmetic composition according to claims 40 wherein the covering index is greater than or equal to 100.

42. A skin makeup kit comprising:
i) a first composition comprising at least one pigment and at least one volatile oil, the first composition having a hue angle h ranging from 40° to 72°;
ii) a second composition having a different color from the first composition, the second composition comprising at least one pigment and at least one volatile oil, the second composition having a hue angle h ranging from 40° to 72°;
wherein the first composition and the second composition have a difference in hue angle Δh ranging from 4° to 40°.

43. A kit according to claim 42 wherein the first composition has a hue angle value h ranging from 40° to 60°.

44. A kit according to claim 43 wherein the first composition has a hue angle value h ranging from 42° to 52°.

45. A kit according to claim 42 wherein the second composition of the kit has a hue angle value h ranging from 54° to 72°.

46. A kit according to claim 45 wherein the second composition of the kit has a hue angle value h ranging from 58° to 72°.

47. A kit according to claim 42 wherein the first composition and the second composition have a difference in hue angle Δh ranging from from 15° to 25°.

48. A skin makeup kit comprising:
i) a first composition comprising at least one pigment and at least one volatile oil, the first composition having a lightness L* ranging from 30 to 80;
ii) a second composition having a different color from the first, the second composition comprising at least one pigment and at least one volatile oil, the second composition having a lightness L* ranging from 30 to 80;
wherein the first composition and second composition have a difference in lightness ΔL* ranging from 2 to 50.

49. A kit according to claim 48 wherein the first composition has a lightness value L* ranging from 30 to 60.

50. A kit according to claim 49 wherein the first composition has a lightness value L* ranging from 40 to 60.

51. A kit according to claim 48 wherein the second composition of the kit has a lightness value L* ranging from 60 to 80.

52. A kit according to claim 48 wherein the second composition of the kit has a lightness value L* ranging from 60 to 70.

53. A kit according to claim 48 wherein the first compositon and second composition have a difference in lightness ΔL* ranging from 20 to 30.

54. A skin makeup kit comprising:
i) a first composition comprising at least one pigment and at least one volatile oil, the first composition having a saturation C* ranging from 10 to 40;
ii) a second composition having a different color from the first, the second composition comprising at least one pigment and at least one volatile oil, the second composition having a saturation C* ranging from 10 to 40;
wherein the first composition and second composition have a difference in saturation ΔC* ranging from 0.1 to 30.

55. A kit according to claim 54 wherein the first composition has a saturation value C* ranging from 10 to 25.

56. A kit according to claim 55 wherein the first composition has a saturation value C* ranging from 20 to 25.

57. A kit according to claim 54 wherein the second composition of the kit has a saturation value C* ranging from 25 to 40.

58. A kit according to claim 57 wherein the second composition of the kit has a saturation value C* ranging from 25 to 31.

59. A kit according to claim 54 wherein the first composition and the second composition have a difference in saturation ΔC* ranging from 5 to 25 .

60. A makeup kit according to claim 42 wherein at least one of the first composition and/or the second composition is an anhydrous cosmetic makeup composition comprising:
i) an oily phase comprising from 10% to 70% by weight, relative to the total weight of the composition, of at least one volatile oil;
ii) a pulverulent phase comprising at least one pigment in an amount greater than or equal to 25% by weight, relative to the total weight of the composition; and
iii) at least one oil-thickening agent;
wherein the compositions have a hardness ranging from 0.04 to 0.5 newtons.

61. A makeup kit according to claim 42 wherein the first composition and the second composition are both anhydrous cosmetic makeup compositions comprising:
i) an oily phase comprising from 10% to 70% by weight, relative to the total weight of the composition, of at least one volatile oil;
ii) a pulverulent phase comprising at least one pigment in an amount greater than or equal to 25% by weight, relative to the total weight of the composition; and
iii) at least one oil-thickening agent;
wherein the compositions have a hardness ranging from 0.04 to 0.5 newtons.

62. A Non-therapeutic skin makeup process comprising applying to the skin at least one anhydrous cosmetic makeup composition comprising:
i) an oily phase comprising from 10% to 70% by weight, relative to the total weight of the composition, of at least one volatile oil;
ii) a pulverulent phase comprising at least one pigment in an amount greater than or equal to 25% by weight, relative to the total weight of the composition; and
iii) at least one oil-thickening agent;
the composition having a hardness ranging from 0.04 to 0.5 newtons.
wherein, when there are at least two compositions they have different colors.

63. A method for effacing and/or covering skin inhomogeneities comprising applying to the skin an anhydrous cosmetic makeup composition comprising:
i) an oily phase comprising from 10% to 70% by weight, relative to the total weight of the composition, of at least one volatile oil;
ii) a pulverulent phase comprising at least one pigment in an amount of greater than or equal to 25% by weight, relative to the total weight of the composition; and
iii) at least one oil-thickening agent;
the composition having a hardness ranging from 0.04 to 0.5 newtons.
